(19) United States
(12) Patent Application Publication
(10) Pub. No.: US 2013/0018368 A1
(43) Pub. Date: Jan. 17, 2013

(54) USER INTERFACE FOR OPERATING AND MONITORING A CRYOSURGICAL SYSTEM

(71) Applicants: Luan Thien Chan, Coon Rapids, MN (US); Satish Ramadhyani, Minneapolis, MN (US); Vineel Vallapureddy, Coon Rapids, MN (US)

(72) Inventors: Luan Thien Chan, Coon Rapids, MN (US); Satish Ramadhyani, Minneapolis, MN (US); Vineel Vallapureddy, Coon Rapids, MN (US)

(73) Assignee: GALIL MEDICAL INC., Arden Hills, MN (US)

(21) Appl. No.: 13/181,810

(22) Filed: Jul. 13, 2011

(51) Int. Cl. A61B 18/02

(52) U.S. Cl. 606/21

(57) ABSTRACT

A method for concurrently displaying on an interactive user interface the various operating parameters and status of the components of a cryosurgical system during a cryosurgical procedure. The interactive user interface enables a cryosurgical system operator to manipulate the operating parameters and reconfigure the display.

### TECHNICAL FIELD

The present invention relates to an interactive user interface for facilitating a cryosurgical procedure.

### BACKGROUND

Known user interfaces for cryosurgical systems are relatively crude in that they are minimally interactive. As such, limited information on the status and progress during a cryosurgical procedure is available to a cryosurgical system operator. Furthermore, the operator is provided a limited number of options for manipulating the operation of the cryoprobes used during the cryosurgical procedure.

Accordingly, there exists a need for a robust interactive user interface which provides the cryosurgical system operator the ability to manipulate the operation of the components comprising the cryosurgical system and to view the progress and status of the procedure.

### SUMMARY

An embodiment of the invention comprises a method for concurrently displaying, on an interactive user interface, the various operating parameters and status of the components of a cryosurgical system during a cryosurgical procedure. The interactive user interface enables a cryosurgical system operator to manipulate the cryoprobe operating parameters such as the mode of operation. Additionally, the operator is provided the ability to select and reconfigure the user interface to concurrently display specific parameters of interest such as the duration of time in different operating modes, temperature profiles, organ specific information, pressures of the cryogas supply sources, time remaining before depletion of the cryogases, etc.

### BRIEF DESCRIPTION OF THE DRAWINGS

FIG. 1 is an embodiment of an interactive user interface for facilitating a cryosurgical procedure;

FIG. 2 is an embodiment of an interactive user interface for manipulating the operating parameters for the cryoprobes;

FIG. 3 is another embodiment of an interactive user interface for facilitating a cryosurgical procedure;

FIG. 4 is yet another embodiment of an interactive user interface for facilitating a cryosurgical procedure; and

FIG. 5 is an alternate view of the interactive user interface of FIG. 1.

### DETAILED DESCRIPTION

While multiple embodiments of the instant invention are disclosed, alternate embodiments may become apparent to a person of ordinary skill in the art. It should be clearly understood that there is no intent, implied or otherwise, to limit the invention in any form or manner to the illustrative embodiments described herein with reference to the accompanying drawings. All alternative embodiments are considered as falling within the spirit, scope and intent of the instant invention.

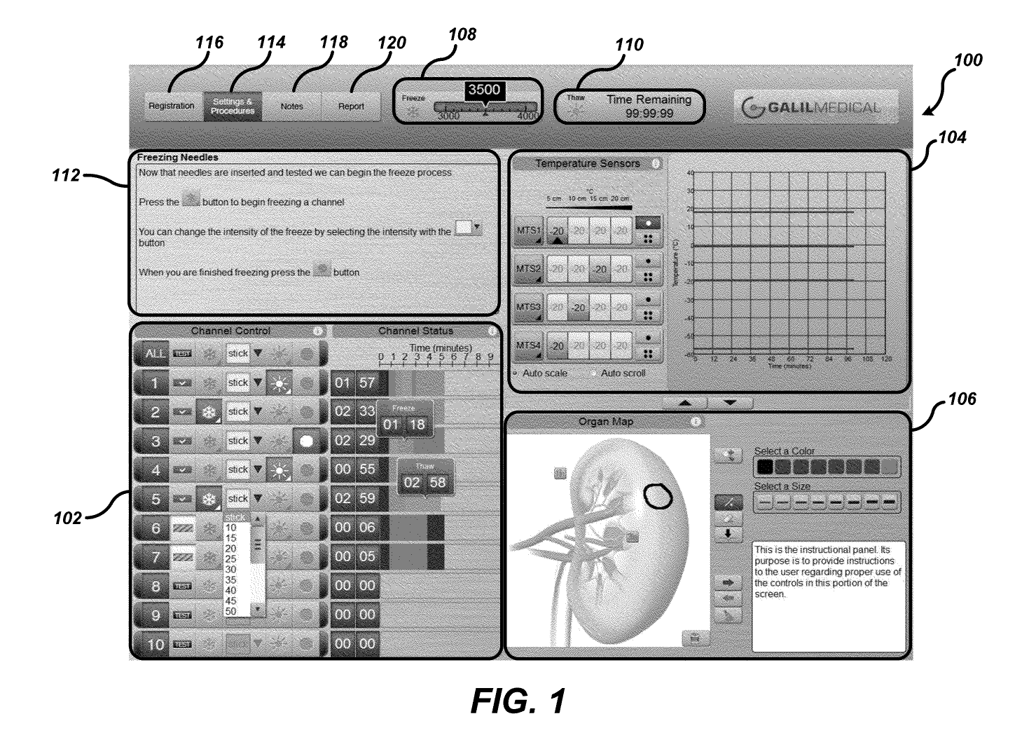

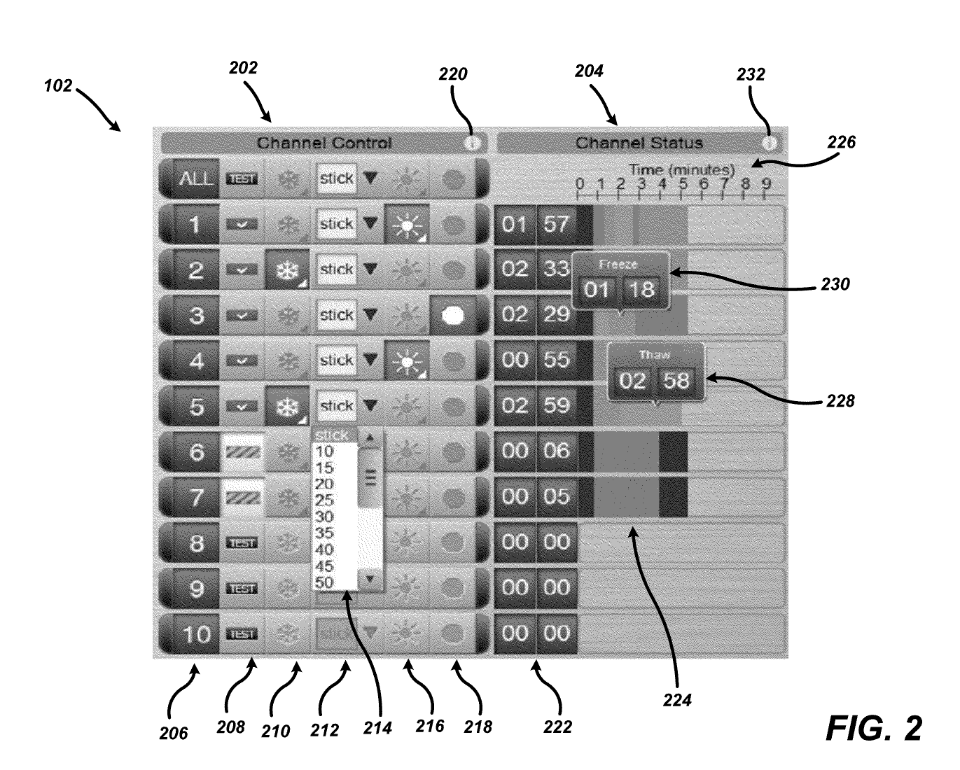

FIG. 1 is a non-limiting embodiment of interactive user interface 100 for facilitating a cryosurgical procedure performed on an organ by an operator of a cryosurgical system. User interface 100 is a touch screen graphical display unit for concurrently displaying operating parameters 102 of a plurality of cryoprobes connected to the cryosurgical system, temperatures 104 within the organ at various distances from the tips of the cryoprobes positioned within the organ, and organ map 106. The cryosurgical system operator's interaction with user interface 100 for viewing and/or changing cryoprobe operating parameters 102, temperatures 104 and organ map 106 is described herein below with reference to FIGS. 2, 3 and 4, respectively.

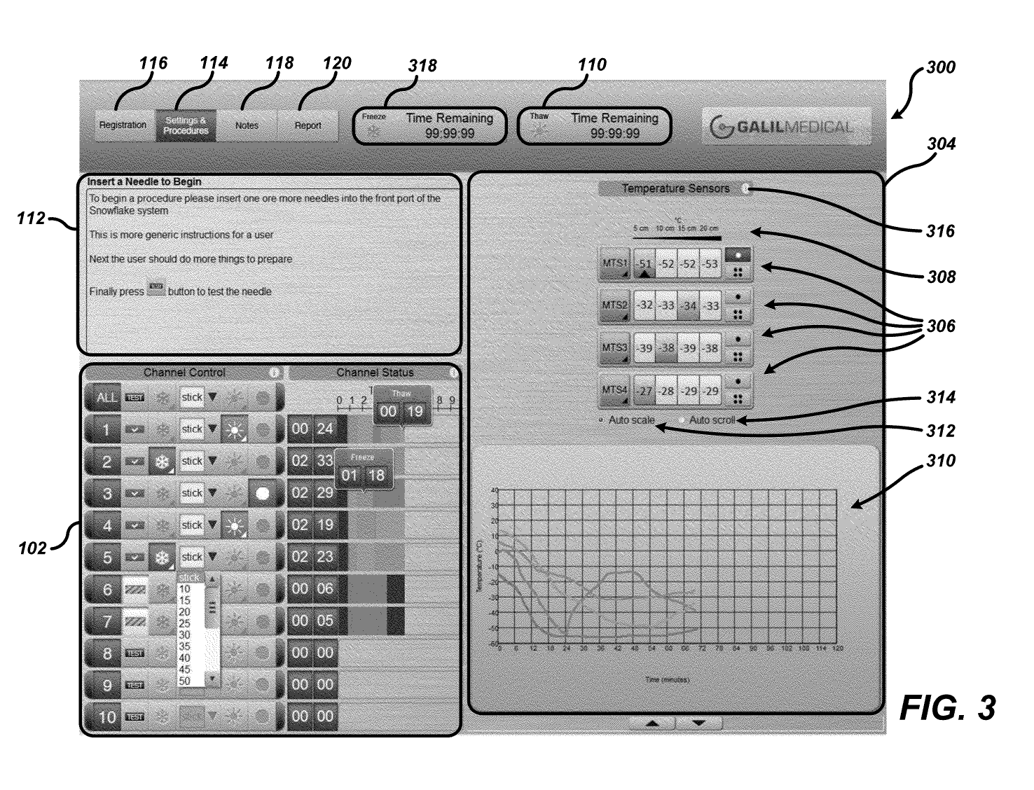

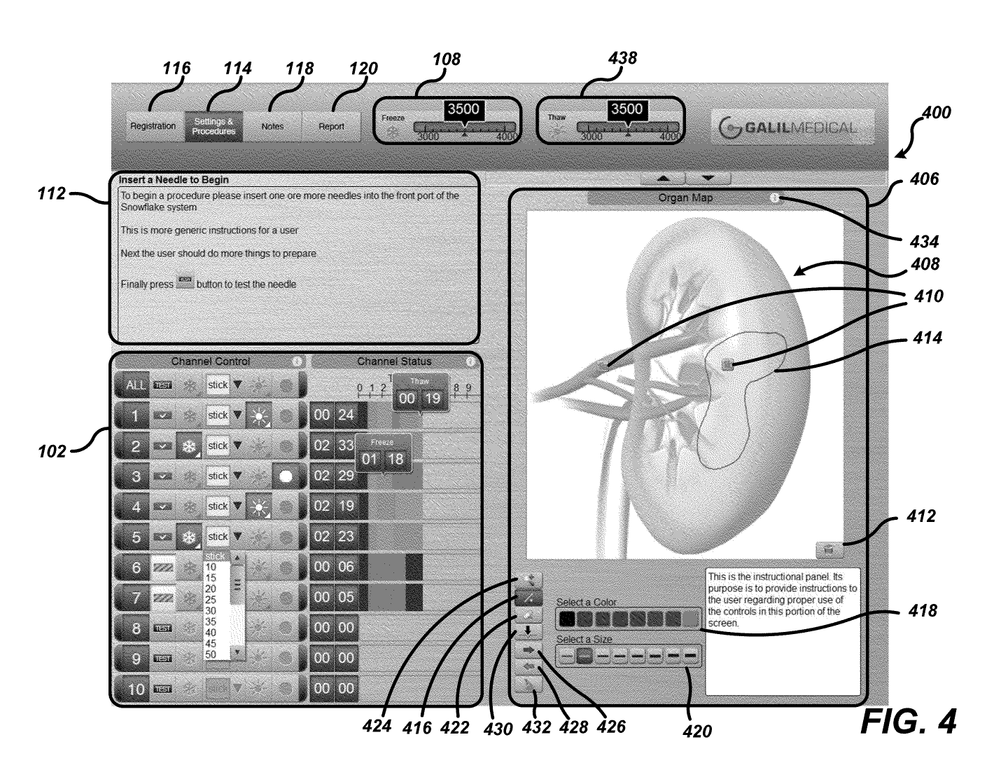

Additionally, user interface 100 alternatively displays the working pressures of the cooling and heating cryogases used by the cryosurgical system, and the time remaining before the sources supplying the cooling and heating cryogases to the cryosurgical system are expended. The exemplary depiction of user interface 100 in FIG. 1 is shown displaying working pressure 108 of the cooling cryogas and time remaining 110 of the heating cryogas. In an embodiment of the invention, the cryosurgical system measures the pressures at which each source, such as a storage tank, supplies the heating and cooling cryogas. Then, based on the number of cryoprobes operating in either the freeze or thaw mode and the heating and cooling gas flow rates for each cryoprobe, the cryosurgical system computes the time remaining before each source supplying the heating and cooling cryogas is anticipated to deplete. In an embodiment of the invention, the cryosurgical system operator manipulates user interface 100 to toggle between displaying the pressure and the time remaining. For instance, by repeatedly selecting, such as by pressing, tapping, clicking, etc., the location on the display unit whereat pressure 108 is displayed, user interface 100 toggles to display the time remaining before the cooling cryogas is anticipated to deplete (see FIG. 3) and working pressure 108 of the cooling cryogas. Similarly, by repeatedly selecting the location on the display unit whereat time remaining 110 is displayed, user interface 100 toggles to display the working pressure of the heating cryogas (see FIG. 4) and time remaining 110 before the heating cryogas is anticipated to deplete.

Furthermore, user interface 100 includes selection tabs labeled “Settings & Procedures” 114, “Registration” 116, “Notes” 118 and “Report” 120.

Selecting “Settings & Procedures” 114 invokes information section 112 for concurrently displaying contextual information to the cryosurgical system operator. In an embodiment of the invention, information section 112 concurrently displays the general and overall state and/or status of the cryosurgical system. Additionally or alternately, information section 112 displays instructions for the operator such as a single or a series of steps in the cryosurgical procedure, information in response to a request for help, warning messages, etc.

Selecting “Registration” 116 enables the user to enter registration information for specifying identification data for the patient, the operator, one or more components of the cryosurgical system such as cryoprobes, etc. Alternately or additionally, selecting “Registration” 116 will concurrently display any registration data that may have been previously entered and/or obtained by the cryosurgical system from other sources.

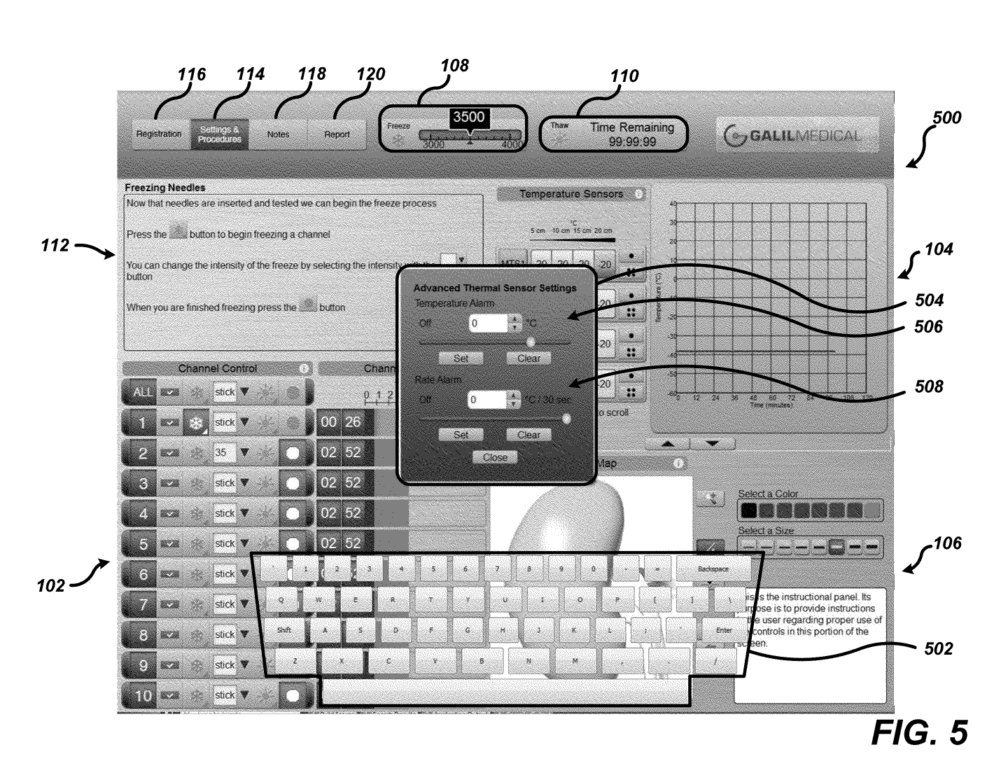

Selecting “Notes” 118 allows the user to enter explanatory text using an input device such as a keyboard, microphone, etc. interfacing with the cryosurgical system. In an embodiment of the invention such as that described herein below with reference to FIG. 5, the keyboard is a virtual keyboard on the display unit of user interface 100. In an alternate embodiment, the keyboard is a physical device interfacing with the cryosurgical system and/or user interface 100. In another embodiment the cryosurgical system and/or user interface 100 includes a speech recognition means for displaying audio notes relayed through the microphone.

Selecting “Report” 120 allows the operator to export a report on the procedure to one or more devices such as a printer, a storage device, a computer or communications network, etc.

It should be clearly understood that FIG. 1 illustrates only one of a plurality of configurations for concurrently displaying information on user interface 100. Some obvious variants include positioning the displayed information in different locations relative to one another. In an alternate embodiment, user interface 100 includes information in addition to or less than or different than that shown by way of example in FIG. 1. In another embodiment of the invention such as that described herein below with reference to FIG. 3, organ map 106 is overlayed or replaced by an enlarged view of temperatures 104 on user interface 100 which concurrently displays all other information described herein above with reference to FIG. 1. In yet another embodiment of the invention such as that described herein below with reference to FIG. 4, temperatures 104 is overlayed or replaced by an enlarged view of organ map 106 on user interface 100 which concurrently displays all other information described herein above with reference to FIG. 1. All variants of any and all information included on user interface 100 and the manner in which such information is displayed on user interface 100 are considered as within the scope, intent, and spirit of the instant invention.

FIG. 2 is an exemplary non-limiting embodiment for concurrently displaying cryoprobe operating parameters 102 on user interface 100, wherein operating parameters 102 comprise channel controls 202 and channel status 204. The term “Channel” as displayed on user interface 100 and as used herein refers to a channel or port associated with the cryosurgical system to which a cryoprobe is connected and through which heating and/or cooling cryogas is supplied to the cryoprobe. In an embodiment of the invention, the channel (or port) is configured for enabling communications between the cryoprobe connected thereto and other components of the cryosurgical system. In one such embodiment, cryoprobe-specific information such as serial number, heating and/or cooling capacities, gas flow rates, etc., encoded within the cryoprobe is accessible by the cryosurgical system through the channel or port. In another embodiment, the cryosurgical system transmits to the cryoprobe through the channel or port to which it is connected. Alternative embodiments are considered as within the scope, intent and spirit of the instant invention.

Channel controls 202, in a non-limiting exemplary embodiment, is an interface through which the operator manipulates the operating parameters of each cryoprobe connected to the cryosurgical system. Channel numbers 206 indicate the total number of ports available in the cryosurgical system, and by extension the number of cryoprobes connected thereto, that can be manipulated, either individually or collectively, by the cryosurgical system operator through user interface 100. As illustrated, channel numbers 1 through 10 are individually associated with a cryoprobe connected to a corresponding port. In an alternate embodiment, the total number of ports available in the cryosurgical system is either more than ten or less than ten. Additionally, the “ALL” channel designation is provided for collectively selecting all the ports in the cryosurgical system such that all the cryoprobes connected thereto are subjected to the same operating parameters. In an embodiment of the invention, channel numbers 206 corresponding to the ports having a cryoprobe connected thereto are conspicuously distinguishable from ports that do not have a cryoprobe connected thereto.

In an embodiment of the invention, the operating parameters that can be manipulated by the cryosurgical system operator comprise the cryoprobe operating mode and the duration of time for operating the cryoprobe in the selected operating mode. Since these operating parameters are available both individually and collectively for all the cryoprobes connected to the ports (or channels) of the cryosurgical system, they will be herein described in their totality and not individually for each cryoprobe. In an embodiment of the invention, the cryoprobe operating mode comprises a test mode, a freeze mode, a stick mode, a thaw mode and a stop (or idle) mode. Additionally, a duration of time is specifiable for the freeze mode and the thaw mode. Each operating parameter, i.e., each operating mode and duration of time, is selected by pressing, pressing and holding, tapping, clicking, etc., a corresponding button or icon or drop-down menu associated therewith. In an embodiment of the invention, a snowflake icon represents the freeze mode, an icon with the text “stick” represents the stick mode, an icon of the sun represents the thaw mode, and a red octagon similar to the stop traffic sign used at road junctions is used to indicate the stop (or idle) mode. Once the operating mode has been selected, and initiated, the conspicuous nature of the corresponding icon is changed, for example to a flashing icon or a different light intensity or color or icon, etc. Alternate icons for representing the operating modes, such as the palm of a hand for the stop mode, and for representing the status of the selected operating mode are considered as within the scope, intent and spirit of the instant invention.

The test mode is initiated by selecting “TEST” in the column designated by reference numeral 208. As illustrated in FIG. 2, the test mode can be initiated either individually for channels 8, 9 and 10 or collectively for “ALL” the cryoprobes connected to the ports of the cryosurgical system. Once the test mode has been initiated, “TEST” is replaced, as shown for channels 6 and 7, by a hashed bar to indicate that the cryoprobe connected to the corresponding port is being tested. In an embodiment of the invention, the hashed bar is dynamic giving the appearance of movement. In an alternate embodiment, an icon other than a hashed bar, for example a stationary or rotating hour glass, is used for indicating that the cryoprobes connected to the corresponding ports are undergoing a test sequence. After the test sequence is successfully completed, the hashed bar is replaced with a check mark icon. In an alternate embodiment, the hashed bar is replaced with an appropriate icon or the text “OK” to indicate a successfully completed test sequence. If the test sequence is not completed successfully, the hashed bar is replaced with an “X” or some other appropriate icon.

In an embodiment of the invention, the test mode is the only mode available when a cryoprobe is first connected to a port. As such, the freeze mode, the stick mode, the thaw mode and the stop mode are not available until after the test sequence has been successfully completed. In an alternate embodiment of the invention, the test mode and the stop mode are the only modes available when a cryoprobe is first connected to a port. As such, the freeze mode, the stick mode and the thaw mode are not available until after the test sequence has been successfully completed. All the modes available before, during and after the testing sequence are identifiable by appropriately conspicuous markings.

The freeze mode is initiated by selecting the snowflake icon in the column designated by reference numeral 210. As illustrated in FIG. 2, the cryoprobes connected to the ports corresponding to channels 2 and 5 are operating in the freeze mode. Also as illustrated in FIG. 2, the snowflake icon comprises an arrow in the lower right corner for indicating that additional options are available for operating the cryoprobes in the freeze mode. These additional options are selectable from a pop up dialog box which is accessed by the continuous and uninterrupted selection of the freeze mode, for example by pressing and holding pressed the snowflake icon 210. One such example is the timed freeze mode which comprises specifying the freeze intensity and the duration of time in the freeze mode. In an alternate embodiment, the additional options are displayed in a different window or screen overlay or a pop-up window.

As illustrated in FIG. 2, the cryoprobes connected to the ports corresponding to channels 1 through 5 are available, either individually or collectively through the “ALL” channel, for freezing and thawing. Upon selecting the downward pointing arrow in the column designated by reference numeral 212, the user is able to manually control the freeze intensity through drop-down menu 214. As shown, drop-down menu 214 includes the “stick” option which enables the user to operate the cryoprobe at a low freeze intensity to produce sufficient ice to stick the needle to the tissue. Alternatively, the user can specify a freeze intensity between 5% and 100% in increments of 5%. While drop-down menu 214 shows freeze intensities selectable between 10% and 50%, the scroll bar between the “up” and “down” arrows on the right side of drop-down menu 214 is used for displaying and selecting freeze intensities different from those shown in FIG. 2. In an alternate embodiment, a different window or screen overlay or pop-up window is used instead of drop down menu 214.

The thaw mode is initiated by selecting the sun icon in the column designated by reference numeral 216. As illustrated in FIG. 2, the cryoprobes connected to the ports corresponding to channels 1 and 4 are operating in the thaw mode. Also as illustrated in FIG. 2, the sun icon comprises an arrow in the lower right corner for indicating that additional options are available for operating the cryoprobes in the thaw mode. These additional options, such as specifying the duration of time in the thaw mode and the thaw intensity including tissue cauterization, are accessed by the continuous and uninterrupted selection of the thaw mode, for example by pressing and holding pressed the sun icon. Although not shown in FIG. 2, the additional options are displayed and selectable from a pop up dialog box. In an alternate embodiment, the additional options are displayed in a different window or screen overlay or pop-up window.

The stop (or idle) mode is initiated by selecting the stop icon in the column designated by reference numeral 218. As will be apparent, the stop mode is selected for terminating any of the other operating modes the cryoprobes may be operating in. As illustrated in FIG. 2, the cryoprobe connected to the port corresponding to channel 3 is in an idle state. Once a port is in the stop mode, i.e., the cryoprobe is in an idle state, it can be used again by selecting any one of the other operating modes.

Information icon 220 provides the cryosurgical system operator access to contextual information pertaining to all aspects of channel control 202 such as those described in the foregoing. In an embodiment of the invention, the information is provided in the form of a pop-up video segment.

Channel status 204, in a non-limiting exemplary embodiment, provides a historical operating status for each cryoprobe connected to the cryosurgical system. As illustrated, the two adjacent columns of numbers designated by reference numeral 222 indicate the total duration of time, in minutes and seconds, the corresponding cryoprobe has been in use. For instance, the respective duration of time that the cryoprobes connected to ports 1, 2 and 4 have been in use are 1 minute and 57 seconds, 2 minute and 3 seconds and 0 minute and 55 seconds. The horizontal bar charts designated by reference numeral 224 are a color coded historical status indicator for the cryoprobe connected to the port represented by corresponding channel numbers 206, wherein each operating mode is represented by a different color. In one embodiment, green represents the test mode, blue represents the freeze mode, yellow represents the thaw mode, and gray represents the stop (or idle) mode. The width of each color coded bar in combination with time axis 226 indicates the duration of time the associated cryoprobe has operated in the operating mode corresponding to that color. For instance a blue bar on bar chart 224 spanning the distance between numerals 2 and 3 on time axis 226 indicates that the associated cryoprobe started operating in the freeze mode 2 minutes after the cryosurgical procedure started, and that the freeze period was 1 minute. Additionally, as illustrated by pop-up windows or overlays 228 and 230, selecting (i.e., pressing, tapping, clicking, etc.) a color coded bar on bar chart 224 numerically displays the duration of time the respective cryoprobe operated in the operating mode corresponding to that color. For instance, selecting the yellow bar from historical bar chart 222 for channel 5 causes pop-up window or overlay 228 to be displayed indicating that specific thaw mode had a duration of 2 minutes and 58 seconds. Similarly, selecting the blue bar from historical bar chart 222 for channel 3 causes pop-up window or overlay 230 to be displayed indicating that specific freeze mode had a duration of 1 minute and 18 seconds.

Information icon 232 provides the cryosurgical system operator access to contextual information pertaining to all aspects of channel status 204 such as those described in the foregoing. In an embodiment of the invention, the information is provided in the form of a pop-up video segment.

Referring back to FIG. 1, interactive user interface 100 is illustrated as concurrently displaying both temperatures 104 and organ map 106. In alternate embodiments of the invention, the cryosurgical system operator is provided the ability to reconfigure user interface 100 to display an enlarged view of either temperatures 104 or organ map 106 concurrently with the other display items such as operating parameters 102, information section 112, etc., described in the foregoing with reference to FIG. 1.

Accordingly, FIG. 3 illustrates interactive user interface 300 as a non-limiting embodiment substantially similar to interactive user interface 100 previously described in reference to FIGS. 1 wherein like reference numerals represent like elements. As shown, an enlarged view of temperatures 304 is concurrently displayed instead of the contracted views of temperatures 104 and organ map 106. In the illustrated embodiment of user interface 300, temperatures 304 concurrently displays a plurality of labeled temperatures 306 at various locations (or distances) 308 within the organ. In an embodiment of the invention, the displayed temperatures indicated by each one of labeled temperatures 306 are measured by temperature sensors positioned at distances 308 from a tip of a temperature probe positioned within the organ. While four labeled temperatures 306 (MTS1, MTS2, MTS3 and MTS4) are shown, alternate embodiments comprising more than four or less than four labeled temperatures 306 are considered as within the scope, intent and spirit of the instant invention.

Additionally, temperatures 304 includes a concurrently displayed historical graph 310 of labeled temperatures 306. While graph 310 is shown displaying four temperature profiles, alternate embodiments of graph 310 comprising more than four or less than four temperature profiles are considered as within the scope, intent and spirit of the instant invention. In an embodiment of the invention, the temperature profiles displayed on graph 310 are from the same distance 308. In an alternate embodiment, the one or more temperature profiles displayed on graph 310 are from different distance 308 selected by the cryosurgical system operator by selecting one or more temperature from labeled temperatures 306. For example, for viewing the historical temperature profile at 5 cm for labeled temperature MTS1, the operator selects the corresponding temperature displayed on labeled temperatures 306. Each one of the one or more temperature selected for inclusion in graph 310 is made conspicuously identifiable on labeled temperatures 306 displayed on user interface 300. For example, as shown in FIG. 3, the temperatures at 5 cm for MTS1, 15 cm for MTS2, 10 cm for MTS3 and 5 cm for MTS4 are conspicuously identifiable on labeled temperatures 306 and are displayed on graph 310. Additional temperature display features such as auto scaling 312 and auto scrolling 314 are also provided. Furthermore, contextual information pertaining to all aspects of temperatures 304 is displayed in information section 112 upon selection of information icon 316.

As previously described in reference to FIG. 1, user interface 100 enables the cryosurgical system operator to toggle between displaying the pressure of the source supplying the cryogas to the cryosurgical system and the time remaining before the source supplying the cryogas is depleted. One such embodiment is illustrated in FIG. 3 wherein user interface 300 concurrently displays time remaining 318 before the source supplying the cooling cryogas to the cryosurgical system is depleted. The concurrently displayed time remaining 318 in user interface 300 toggles with pressure 108 in user interface 100.

Referring back to FIG. 1, interactive user interface 100 is illustrated as concurrently displaying both temperatures 104 and organ map 106. In alternate embodiments of the invention, the cryosurgical system operator is provided the ability to reconfigure user interface 100 to display an enlarged view of either temperatures 104 or organ map 106 concurrently with the other display items such as operating parameters 102, information section 112, etc. described in the foregoing with reference to FIG. 1.

Accordingly, FIG. 4 illustrates interactive user interface 400 as a non-limiting embodiment substantially similar to interactive user interface 100 previously described in reference to FIG. 1 wherein like reference numerals represent like elements. As shown, an enlarged view of organ map 406 is concurrently displayed instead of the contracted views of temperatures 104 and organ map 106. In the illustrated embodiment of user interface 400, organ map 406 concurrently displays an enlarged view of organ 408 on which the cryosurgical procedure is conducted. In an embodiment of the invention, the cryosurgical system operator places cryoprobe icons 410 on organ 408 to indicate an approximate location whereat one or more cryoprobe has been positioned within the organ. Cryoprobe icons 410 are removed from organ 408 by dragging and dropping into wastebasket (or thrash can) 412. Alternatively, cryoprobe icons 410 are removed by the operator sequentially selecting one cryoprobe icon 410 and wastebasket 412 in any order. In an alternate embodiment, the cryosurgical system displays or removes cryoprobe icons 410 based on information obtained from an ultra-sound, MRI, etc. In another embodiment of the invention, icons similar to cryoprobe icons 410 are used for indicating an approximate location whereat one or more temperature probe is positioned within the organ. User interface 400 further enables the operator to identify one (or more) section 414 on organ 408 by selecting pencil 416, color palette 418 and size 420. Erasure 422 is provided for removing section 414 from organ 408. Additionally, the operator uses icon (e.g., a magnifying glass or other icon) 424 to select a different organ 408 as viewed in organ map 406. Alternate views of organ 408, such as different orientations, are concurrently displayed in organ map 406 (and by extension on user interface 400) by selecting orientation icons 426, 428 and 430. Paint brush 432 in combination with color palette 418 and size 420 is used for marking one or more locations on organ 408. In an embodiment of the invention, paint brush 432 and color palette 418 are used in combination to color the section of the organ within the boundary of section 414. Upon selecting information icon 434, contextual information pertaining to all aspects of organ map 406 is accessible to the cryosurgical system operator. In an embodiment of the invention, the information is provided in the form of a pop-up video segment Additionally, information section 112 on user interface 400 is also available for displaying contextual information.

As previously described in reference to FIG. 1, user interface 100 enables the cryosurgical system operator to toggle between displaying the pressure of the source supplying the cryogas to the cryosurgical system and the time remaining before the source supplying the cryogas is depleted. One such embodiment is illustrated in FIG. 4 wherein user interface 400 concurrently displays pressure 438 of the source supplying heating cryogas to the cryosurgical system. The concurrently displayed pressure 438 in user interface 400 toggles with time remaining 110 in user interface 100.

Referring now to FIG. 5, interactive user interface 500 is illustrated comprising a pop-up virtual keyboard 502 concurrently overlayed (or superimposed) on the display. Selecting “Notes” 118 such as by pressing, tapping, clicking, etc., concurrently displays virtual keyboard 502 which is used by the cryosurgical system operator for entering notes before, during, or after the cryosurgical procedure.

Referring back to FIG. 3, labeled temperatures 306 are illustrated having an arrow in the lower right corner of the labels MTS1, MTS2, MTS3 and MTS4 indicating that additional options are available. In an embodiment of the invention, such additional options comprise providing the operator the ability to set advanced settings 504. As shown in FIG. 5, advanced settings 504 is concurrently displayed as a pop-up window overlayed (or superimposed) on user interface 500. In an embodiment of the invention, the cryosurgical system operator sets or clears temperature alarm 506 and sets or clears temperature rate alarm 508. If temperature alarm 506 is set, the operator is alerted when the organ temperature decreases below the specified value. Rate alarm 508 is used by the operator for being alerted when the organ temperature changes at a rate greater than the specified rate.

It should be clearly understood that the various embodiments described in the foregoing illustrate only some configurations for concurrently displaying information on a user interface. Some obvious variants include positioning the displayed information in different locations relative to one another. In alternate embodiments, the user interface includes information in addition to or less than or different than that shown by way of example in FIGS. 1-5. All variants of any and all information included on a user interface and the manner in which such information is displayed on a user interface are considered as within the scope, intent, and spirit of the instant invention.

Various modifications and additions may be made to the exemplary embodiments presented hereinabove without departing from the spirit, scope and intent of the present invention. For example, while the disclosed embodiments refer to particular features, the scope of the instant invention is considered to also include embodiments having various combinations of features different from and/or in addition to those described hereinabove. Accordingly, the present invention embraces all such alternatives, modifications, and variations as within the spirit, scope and intent of the appended claims, including all equivalents thereof.

We claim:

1. A method of facilitating a cryosurgical procedure, comprising
positioning a distal end of a cryoprobe within an organ;
positioning a temperature sensor within the organ; and
concurrently displaying on a user interface
operating parameters for the cryoprobe;
a temperature measured by the temperature sensor;
a graph of the temperature measured over a period of time;
a pressure of a cryogas source;
an amount of time remaining before depletion of the cryogas source; and
contextual information for an operator performing the cryosurgical procedure.

2. The method of claim 1, wherein said operating parameters comprise
a test mode;
a freeze mode;
a duration of time for the freeze mode;
a stick mode;
a thaw mode, said thaw mode including a cauterization mode;
a duration of time for the thaw mode; and
a stop mode.

3. The method of claim 2, comprising concurrently displaying on the user interface an actual duration of time the cryoprobe has operated in the test mode, the freeze mode, the thaw mode, and an idle state.

4. The method of claim 1, comprising concurrently using the user interface for changing said operating parameters during the cryosurgical procedure.

5. The method of claim 1, further comprising enunciating an alarm when the measured temperature is greater than or less than pre-specified values or changes more than a pre-specified value over a pre-specified period of time.

6. The method of claim 1, further comprising concurrently displaying on the user interface
a graphical representation of the organ; and
an approximate location of the distal end of the cryoprobe on the graphical representation of the organ.

7. The method of claim 6, wherein the graphical representation of the organ is displayed in alternate orientations.

8. The method of claim 6, wherein said graphical representation of the organ is an enlarged graphical representation of the organ, said enlarged graphical representation of the organ replacing the displayed temperature.

9. The method of claim 8, wherein the enlarged graphical representation of the organ is displayed in alternate orientations.

10. The method of claim 6, comprising removing the distal end of the cryoprobe from the graphical representation of the organ.

11. A method of facilitating a cryosurgical procedure, comprising
positioning a distal end of a cryoprobe within an organ; and
concurrently displaying on a user interface
a graphical representation of the organ;
an approximate location of the distal end of the cryoprobe on the graphical representation of the organ;
operating parameters for the cryoprobe;
a pressure of a cryogas source; and
contextual information for an operator performing the cryosurgical procedure.

12. The method of claim 11, wherein said operating parameters comprise
a test mode;
a freeze mode;
a duration of time for the freeze mode;
a stick mode;
a thaw mode, said thaw mode including a cauterization mode;
a duration of time for the thaw mode; and
a stop mode.

13. The method of claim 12, comprising concurrently displaying on the user interface an actual duration of time the cryoprobe has operated in the test mode, the freeze mode, the thaw mode, and an idle state.

14. The method of claim 11, comprising concurrently using the user interface for changing said operating parameters during the cryosurgical procedure.

15. The method of claim 11, further comprising concurrently displaying on the user interface an amount of time remaining before depletion of the cryogas source.

16. The method of claim 11, comprising displaying alternate views of the graphical representation of the organ, said alternate views including a plurality of magnifications and a plurality of orientations.

17. The method of claim 11, comprising removing the distal end of the cryoprobe from the graphical representation of the organ.

18. A method of facilitating a cryosurgical procedure, comprising
positioning a distal end of a cryoprobe within an organ;
positioning a temperature sensor within the organ; and
concurrently displaying on a user interface
operating parameters for the cryoprobe;
a temperature measured by the temperature sensor;
a pressure of a cryogas source; and
contextual instructions for an operator performing the cryosurgical procedure.

19. The method of claim 18, wherein said operating parameters comprise
a test mode;
a freeze mode;
a duration of time for the freeze mode;
a stick mode;
a thaw mode, said thaw mode including a cauterization mode;
a duration of time for the thaw mode; and
a stop mode.

20. The method of claim 19, comprising concurrently displaying on the user interface an actual duration of time the cryoprobe has operated in the test mode, the freeze mode, the thaw mode, and an idle state.

21. The method of claim 18, comprising concurrently using the user interface for changing said operating parameters during the cryosurgical procedure.

22. The method of claim 18, further comprising concurrently displaying on the user interface an amount of time remaining before depletion of the cryogas source.

23. The method of claim 18, comprising concurrently displaying on the user interface a graph of the temperature measured over a period of time.

24. The method of claim 18, further comprising enunciating an alarm when the measured temperature is greater than or less than pre-specified values or changes more than a pre-specified value over a pre-specified period of time.
